(19) United States
(12) Patent Application Publication
(10) Pub. No.: US 2008/0145316 A1
(43) Pub. Date: Jun. 19, 2008

(54) Skin coating with microbial indicator

(76) Inventors: John Gavin MacDonald, Decatur, GA (US); Stephanie Martin, Woodstock, GA (US); Molly K. Smith, Atlanta, GA (US); Jason Lye, Atlanta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/639,833

(22) Filed: Dec. 14, 2006

(51) Int. Cl. A61K 49/00; C12Q 1/04; C12Q 1/70

(52) U.S. Cl. 424/10.3; 424/10.1; 435/34; 435/5

(57) ABSTRACT

Skin sealants are usually applied over skin preps to seal the skin and hold any remaining bacteria in place prior to surgical incisions. This sealant is generally left on the skin after surgery. A skin coating is provided that has an indicator that gives a visible color change upon contact with microbes or microbial by-products and so provides an early warning of infection. The coating is a curable coating composition that may also be used without skin preps and may be used to protect other disruptions in the skin like wounds, bruises, abrasions, burns, acne, blisters, bites, stings, punctures and cuts. It may also be used to close wounds or provide an additional barrier to other parts of the skin, such as the nails and mucosa.

### BACKGROUND OF THE INVENTION

Surgical site infections (SSI) occur following about 2-3 percent of surgeries in the United States with an estimated 500,000 incidents of SSI occurring annually, which can lead to significant patient morbidity and mortality. In addition to the negative impact of such infections on patient health, these potentially avoidable infections contribute significantly to the financial burden experienced by the health care system. SSIs result when an incision becomes contaminated by bacteria, and for most surgeries the primary source of these infection-causing microorganisms is the skin (an exception being surgeries in which the gastrointestinal tract is penetrated).

Various compositions are used to prepare the skin prior to surgery. Skin preparations or “preps” are used to remove some level of microbial load on the skin prior to making an incision. Skin sealant materials are used to protect patients from bacterial infections associated with surgical site incisions and insertion of intravenous needles. Skin preps are applied to the skin and allowed to dry to maximize effectiveness for reducing microorganisms. After the skin prep has dried, the sealant may be applied directly to the skin in liquid form. The sealant forms a coherent film with strong adhesion to the skin through various techniques based on the chemistry of the sealant composition.

Skin preps currently are predominantly povidone-iodine or chlorhexidine gluconate based formulations and may contain alcohol for fast drying and more effective killing of organisms

Skin sealants now use a polymer composition that dries to form a film through evaporation of a solvent, for example. Other skin sealants contain monomeric units that polymerize in situ to from a polymeric film. Cyanoacrylate sealants containing alkyl cyanoacrylate monomer are an example of the latter type wherein the monomer polymerizes in the presence of a polar species such as water or protein molecules to form an acrylic film. The resulting film formed serves to immobilize bacterial flora found on the skin and prevents their migration into an incision made during a surgical procedure or skin puncture associated with insertion of an intravenous needle.

A skin coating may also encompass substances designed to protect or treat the nails or mucosal surfaces of the body. Such substances include nail polish, eyedrops, nasal sprays, etc and serve to provide an additional barrier between the skin and the environment.

While the use of skin sealants has significantly reduced the occurrence of surgical site infections, they remain a great concern. There is currently no known skin sealant that will indicate when microbial contamination is present. Such an indicator would give the medical provider an early warning to the presence of an infection or the possibility of such an infection developing.

It is clear that there exists a need for an indicator of microbial contamination for use in surgical applications.

### SUMMARY OF THE INVENTION

In response to the foregoing difficulties encountered by those of skill in the art, we have discovered a novel subset of dyes and colorants that may be successfully added to skin coatings to visibly indicate the presence of microbes that may lead to infection. Some of the dyes have a response to a broad spectrum of microbes while others are specific to particular yeasts, bacteria, molds and/or viruses. The indicator may be present in the coating composition in an amount less than or equal to about 1000 parts per million (ppm), more particularly between 50 and 800 ppm and still more particularly between 100 and 500 ppm. The curable coating and indicator could be used to verify skin cleanliness prior to surgery, and should show the presence of microbes in a time of less than 20 minutes after contact, more particularly less than 5 minutes after contact with the microbes and still more particularly less than 30 seconds after contact. Conversely, the curable coating and indicator could be used to monitor the build up of microbial contamination on the skin surface over time. The microbes could be already present, in the or on the skin, in very small amounts and with time multiply to form a colony with sufficient number that a serious infection would result. They could also come from contamination after surgery through contact with infected hands, instruments or needles etc. The microbial contamination indicating coating would be able to detect either case; such as instant contamination of a high number of microbes present or the build-up of microbes on or in the skin over time.

### DETAILED DESCRIPTION OF THE INVENTION

It has been discovered that microorganism contamination may be detected through the use of a dye or colorant that produces a distinct spectral response for a microorganism or class or microorganisms. The microorganisms that may be detected are not particularly limited, and may include bacteria, yeast, fungi, mold, protozoa, viruses, etc. Several relevant bacterial groups that may be detected include, for instance, gram negative rods (e.g., Entereobacteria); gram negative curved rods (e.g., vibious, Heliobacter, Campylobacter, etc.); gram negative cocci (e.g., Neisseria); gram positive rods (e.g., Bacillus, Clostridium, etc.); gram positive cocci (e.g., Staphylococcus, Streptococcus, etc.); obligate intracellular parasites (e.g., Ricckettsia and Chlamydia); acid fast rods (e.g., Myobacterium, Nocardia, etc.); spirochetes (e.g., Treponema, Borellia, etc.); and mycoplasmas (i.e., tiny bacteria that lack a cell wall). Particularly relevant bacteria include E. coli (gram negative rod), Klebsiella pneumonia (gram negative rod), Streptococcus (gram positive cocci), Salmonella choleraesuis (gram negative rod), Staphyloccus aureus (gram positive cocci), and P. aeruginosa (gram negative rod).

In addition to bacteria, other microorganisms of interest include molds and yeasts (e.g., Candida albicans), which belong to the Fungi kingdom. Zygomycota, for example, is a class of fungi that includes black bread mold and other molds that exhibit a symbiotic relationship with plants and animals. These molds are capable of fusing and forming tough “zygospores.” Ascomycota is another class of fungi, which includes yeasts, powdery mildews, black and blue-green molds, and some species that cause diseases such as Dutch elm disease, apple scab, and ergot. The life cycle of these fungi combines both sexual and asexual reproduction, and the hyphae are subdivided into porous walls that allow for passage of the nuclei and cytoplasm. Deuteromycota is another class of fungi that includes a miscellaneous collection of fungi that do not fit easily into the aforementioned classes or the Basidiomycota class (which includes most mushrooms, pore fungi, and puffball fungi). Deuteromycetes include the species that create cheese and penicillin, but also includes disease-causing members such as those that lead to athlete's foot and ringworm. More specifically, athlete's foot (also called tinea pedis) is caused by the ring worm fungus tinea. Upto 70% of the population will have athlete's foot infection at some time during their lives. It is spread from person to person by contact with infected floor, socks and clothing. Nail fungus (onychomycosis) can infect fingernails and toenails and is very common. More than 35 million people in the United States have it under their nails. It is commonly passed from human to human via shower stalls, bathrooms, or locker rooms where people move around with bare feet.

The term “skin” as used herein, means all external surface areas of the body including nails, hair, skin, eyes, mucosal membranes. The skin proper consists of three layers: epidermis, dermis and subcutaneous tissue. This indicator would be able to detect microbial contamination or infection present on or in the first two layers through contact with either the microbes themselves or associated by-products such as volatiles, metabolites, or other microbe-associated elements.

Skin sealant materials are curable coatings used to protect patients from bacterial infections associated with surgical site incisions and insertion of intravenous needles. Skin sealants are often applied directly over or on top of (Betadine®) skin preps. The sealant forms a coherent film with strong adhesion to the skin through various techniques based on the chemistry of the sealant composition. Skin sealants such as cyanoacrylate sealants containing alkyl cyanoacrylate monomer are an example of the type wherein the monomer polymerizes in the presence of a polar species such as water or protein molecules to form an acrylic film. Cyanoacrylates include, for example, a 2-alkyl cyanoacrylate where the alkyl group is a C₁ to C₈ hydrocarbon which is straight chain, branched chain, or cyclic.

It would be useful for medical personnel to have as early a warning as possible to microbial infection of an incision or other type of skin wound. The inventors believe that providing a skin coating that will change color in the presence of microbes will provide valuable information for the medical professional.

Initially it was thought that placing one of the microbial indicators into a skin coating formulation would not allow the indicating dye to be in contact with the microbe causing the infection or contamination and therefore would not trigger a visual indication. This lack of activity would be due to the majority of the dye being retained in the bulk of the skin coating and therefore not on the skin/coating interface. The diligent work of the inventors showed, however, that there is sufficient dye present on the film surface to give a visual color change when in the presence of microbial contamination. Though not wishing to be bound by this speculation, the inventors believe that the dye appears to be concentrated towards the surface of the film due in part to the crystallization of the polymer during curing and also due to the surface segregation of the dye due to the slight incomparability of the dye in the polymer.

In addition to being used as a traditional skin sealant, i.e. as a film forming barrier through which a surgical incision is made, the indicator and curable coating composition may also be used like a bandage to close and/or cover wounds, bruises, abrasions, burns, acne, blisters, bites, stings, nails, cuticles, punctures, cuts and other disruptions in the skin to protect them from subsequent contamination or indicate the presence due to growth of precontamination areas. The use of the skin coating composition would therefore not be limited to medical personnel and would not require the use of a skin prep before the skin coating is applied.

Wound protection is critical in permitting the healing process to take place. Traditional adhesive bandages and gauze wound dressings have been used by the consumer to treat/dress acute wounds or skin irritations. Such adhesive bandages are generally passive, in that they offer little or no chemical treatment for wound healing. Rather, they primarily serve to exert low levels of pressure on the wound, protect the wound from exposure to the environment, and absorb any exudates, which are produced from the wound site. Such bandages generally include a base layer, which is the layer seen by the consumer following application of the bandage to the wound. Such a layer is typically formed from a polymeric material such as a film, nonwoven web, or combination thereof, and may be perforated in some fashion to allow for flexibility and/or further breathability. This layer often includes a film component, having a top side surface which is seen by the consumer after application of the bandage to the wound site, and a bottom side surface (skin contacting surface). A skin-friendly adhesive is usually placed over the base layer bottom side surface to provide a means for attaching the bandage to the consumer. Alternatively, a separate adhesive tape is used to attach the bandage/wound dressing to the wound site, if the bandage/wound dressing is of the nonadhesive type. In the center of the base layer bottom side surface is traditionally positioned an absorbent pad for absorbing exudates from the wound. Finally, a non-stick perforated film layer is normally positioned over the absorbent pad layer, to provide a barrier between the absorbent pad and the wound itself. This allows the wound fluid to move through the perforated layer without sticking to the wound site. Typically the absorbent pad in such bandage does not include any medicinal components, although comparatively recently, bandage manufacturers have started including antibiotic agents on or within bandages to encourage wound healing.

The skin coating composition of this invention can replace this seemingly complicated bandage construction with a single liquid treatment that will dry to a flexible coating that protects a wound much like a bandage would. Additionally, medicaments such as antibiotic agents may be blended in effective amounts with the composition to provide additional benefits in the area of microbial inhibition and the promotion of wound healing. The coating may be applied to provide an effectively thick coating over the surface of the superficial wound, burn or abrasion. Because the to-be-treated wound is superficial and does not extend beyond the dermal layer, any polymeric residues diffusing into or forming in the wound will be naturally extruded from the skin. Generally, the coating provides an adhesive film coating over the wound area which when set is satisfactorily flexible and adherent to the tissue without premature peeling or cracking. The coating generally has a thickness of less than about 0.5 millimeter (mm).

Sealant coatings of such thicknesses form a physical barrier layer over superficial wounds which provide protection for the wound in the same manner as a conventional bandage. Specifically, the coating provides an almost airtight, waterproof seal around the wound which does not need to be replaced when the wound gets wet. Once applied, the coating prevents bacterial and contaminant entry into the wound, thus reducing the rate of secondary infection. Generally, the adhesive coating does not limit dexterity and promotes faster wound healing. Additionally, unlike conventional bandages, the coating naturally sloughs off the skin within 2-3 days after application and, accordingly, avoids the discomfort associated with removal of conventional bandages from the skin. However, if early removal of this polymeric coating is desired, such can be achieved by use of solvents such as acetone. Further discussion of this use may be found in U.S. Pat. No. 6,342,213.

By way of elaboration it should be noted that several wound care products are currently being marketed which contain an antiseptic benzalkonium chloride and an antibiotic mixture of polymixin B-sulfate and bacitracin-zinc. Patents in this area of technology have described the use of commonly known antiseptics and antibiotics, such as those described in U.S. Pat. Nos. 4,192,299, 4,147,775, 3,419,006, 3,328,259, and 2,510,993. U.S. Pat. No. 6,054,523, to Braun et al., describes materials that are formed from organopolysiloxanes containing groups that are capable of condensation, a condensation catalyst, an organopolysiloxane resin, a compound containing a basic nitrogen, and polyvinyl alcohol. U.S. Pat. No. 5,112,919, reported a moisture-crosslinkable polymer that was produced by blending a thermoplastic base polymer, such as polyethylene, or a copolymer of ethylene, with 1-butene, 1-hexene, 1-octene, or the like; a solid carrier polymer, such as ethylene vinylacetate copolymer (EVA), containing a silane, such as vinyltrimethoxysilane; and a free-radical generator, such as an organic peroxide; and heating the mixture. The copolymers could then be cross-linked by reaction in the presence of water and a catalyst, such as dibutyltin dilaurate, or stannous octoate. U.S. Pat. No. 4,593,071 to Keough reported moisture cross-linkable ethylene copolymers having pendant silane acryloxy groups.

A polyurethane wound coating is described by Tedeshchl et al., in EP 0992 252 A2, where a lubricious, drug-accommodating coating is described that is the product of a polyisocyanate; an amine donor, and/or a hydroxyl donor; and an isocyanatosilane adduct having terminal isocyanate groups and an alkoxy silane. A water soluble polymer, such as poly(ethylene oxide), can optionally be present. Cross-linking causes a polyurethane or a polyurea network to form, depending upon whether the isocyanate reacts with the hydroxyl donors or the amine donors. U.S. Pat. No. 6,967,261 describes the use of chitosan in wound treatment. Chitosan is a deacetylated product of chitin (C₈H₁₃NO₅)ₙ, an abundant natural glucosamine polysaccharide. In particular, chitin is found in the shells of crustaceans, such as crabs, lobsters and shrimp. The compound is also found in the exoskeletons of marine zooplankton, in the wings of certain insects, such as butterflies and ladybugs, and in the cell wall of yeasts, mushrooms and other fungi. Antimicrobial properties of chitosan have been reported against Gram positive and Gram negative bacteria, including Streptococcus spp., Staphylococcus aureus, Staphylococcus epidermidis, Staphylococcus haemolyticus, Pseudomonas, Escherichia, Proteus, Klebsiella, Serratia, Acinobacter, Enterobacter and Citrobacter spp. Chitosan has also been described in the literature to induce repair of tissue containing regularly arranged collagen bundles.

The composition may also be used to close wounds much like stitches or bandages. To be used in such a way, the composition is applied to at least one skin surface of the opposed skin sections of, for example, a suturable wound of a mammalian patient (e.g., human patient). The opposed skin sections are contacted with each either before or after application of the composition. In either case, after application of the composition, the wound area is maintained under conditions wherein the composition polymerizes to join these skin sections together. In general, a sufficient amount of the composition may be employed to cover the wound and the adjacent the skin surface of at least one of the opposed skin sections of the suturable wound. Upon contact with skin moisture and tissue protein, the composition will polymerize or, in the case of compositions utilizing partially polymerized monomers, will further polymerize, at ambient conditions (skin temperature) over about 10 seconds to 60 seconds to provide a solid polymeric film which joins the skin sections, thereby closing the wound. Generally, the composition can provide a polymeric film over the separated skin sections thereby inhibiting infection of the wound while promoting healing. Further discussion of this use may be found in U.S. Pat. No. 6,214,332.

The coating composition may also be used to cover the nails and mucosal membranes. The microbial indicating dye may be added to various drops, gels, nail polishes and the like to indicate the presence of fungal infections. Nail fungus (onychomycosis) can infect fingernails and toenails and is very common. A common treatment for onychomycosis is to coat the suspect nail with a topical solution of 8% ciclopirox solution, commonly available under the trade name “Penlac”. The indicator may be added, for example, to Penlac® lacquer, (ciclopirox), to indicate the location of nail fungus. The indicator may likewise be added to common nail polish.

Suitable dyes or colorants capable of exhibiting a color change in the presence of one or more microorganisms have already been described in US patent application 20060134728 by MacDonald et. al and US 20050130253 by MacDonald et. al which are incorporated in there entirety herein. As described the colorant may change from a first color to a second color, from colorless to a color, or from a color to colorless. A variety of colorants (e.g., dyes, pigments, etc.) may be employed in the practice of the present invention, the structures of some of which are given in Table 1. In one embodiment, for example, pH-sensitive colorants are employed that are capable of differentiating between certain types of microorganisms. Namely, pH-sensitive colorants can detect a change in the pH of the growth medium of the microorganism. Bacteria, for instance, may metabolize the growth medium and generate acidic compounds (e.g., CO₂) that lead to a change in pH. Likewise, certain microorganisms (e.g., bacteria) contain highly organized acid moieties on their cell walls. Because the acidic/basic shift may vary for different microorganisms, pH-sensitive colorants may be selected in the present invention that are tuned for the desired pH transition. It is also possible to include a non-indicating color dye with the at least one microbial indicator.

Phthalein colorants constitute one class of suitable pH-sensitive colorants that may be employed in the array of the present invention. Phenol Red (i.e., phenolsulfonephthalein), for example, exhibits a transition from yellow to red over the pH range 6.6 to 8.0. Above a pH of about 8.1, Phenol Red turns a bright pink (fuschia) color. Derivatives of Phenol Red may also be suitable for use, such as those substituted with chloro, bromo, methyl, sodium carboxylate, carboxylic acid, hydroxyl and amine functional groups. Exemplary substituted Phenol Red compounds include, for instance, Chlorophenol Red, Metacresol Purple (meta-cresolsulfonephthalein), Cresol Red (ortho-cresolsulfonephthalein), Pyrocatecol Violet (pyrocatecolsulfonephthalein), Chlorophenol Red (3′,3″-dichlorophenolsulfonephthalein), Xylenol Blue (the sodium salt of para-xylenolsulfonephthalein), Xylenol Orange, Mordant Blue 3 (C.I. 43820), 3,4,5,6-tetrabromophenolsulfonephthalein, Bromoxylenol Blue, Bromophenol Blue (3′,3″,5′,5″-tetrabromophenolsulfonephthalein), Bromochlorophenol Blue (the sodium salt of dibromo-5′,5″-dichlorophenolsulfonephthalein), Bromocresol Purple (5′,5″-dibromo-ortho-cresolsulfonephthalein), Bromocresol Green (3′,3″,5′,5″-tetrabromo-ortho-cresolsulfonephthalein), and so forth. Still other suitable phthalein colorants are well known in the art, and may include Bromothymol Blue, Thymol Blue, Bromocresol Purple, thymolphthalein, and phenolphthalein (a common component of universal indicators). For example, Chlorophenol Red exhibits a transition from yellow to red over a pH range of about 4.8 to 6.4; Bromothymol Blue exhibits a transition from yellow to blue over a pH range of about 6.0 to 7.6; thymolphthalein exhibits a transition from colorless to blue over a pH range of about 9.4 to 10.6; phenolphthalein exhibits a transition from colorless to pink over a pH range of about 8.2 to 10.0; Thymol Blue exhibits a first transition from red to yellow over a pH range of about 1.2 to 2.8 and a second transition from yellow to pH over a pH range of 8.0 to 9.6; Bromophenol Blue exhibits a transition from yellow to violet over a pH range of about 3.0 to 4.6; Bromocresol Green exhibits a transition from yellow to blue over a pH range of about 3.8 to 5.4; and Bromocresol Purple exhibits a transition from yellow to violet over a pH of about 5.2 to 6.8.

Hydroxyanthraquinones constitute another suitable class of pH-sensitive colorants. Hydroxyanthraquinones have the following general structure:

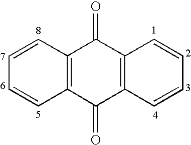

The numbers 1-8 shown in the general formula represent a location on the fused ring structure at which substitution of a functional group may occur. For hydroxyanthraquinones, at least one of the functional groups is or contains a hydroxy (—OH) group. Other examples of functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, phosphorous groups, etc. Some suitable hydroxyanthraquinones that may be used in the present invention, Mordant Red 11 (Alizarin), Mordant Red 3 (Alizarin Red S), Alizarin Yellow R, Alizarin Complexone, Mordant Black 13 (Alizarin Blue Black B), Mordant Violet 5 (Alizarin Violet 3R), Alizarin Yellow GG, Natural Red 4 (carminic acid), amino-4-hydroxyanthraquinone, Emodin, Nuclear Fast Red, Natural Red 16 (Purpurin), Quinalizarin, and so forth. For instance, carminic acid exhibits a first transition from orange to red over a pH range of about 3.0 to 5.5 and a second transition from red to purple over a pH range of about 5.5 to 7.0. Alizarin Yellow R, on the other hand, exhibits a transition from yellow to orange-red over a pH range of about 10.1 to 12.0.

Yet another suitable class of pH-sensitive colorants that may be employed is aromatic azo compounds having the general structure:

X—R₁—N═N—R₂—Y

wherein,

R₁ is an aromatic group;

R₂ is selected from the group consisting of aliphatic and aromatic groups; and

X and Y are independently selected from the group consisting of hydrogen, halides, —NO₂, —NH₂, aryl groups, alkyl groups, alkoxy groups, sulfonate groups, —SO₃H, —OH, —COH, —COOH, halides, etc. Also suitable are azo derivatives, such as azoxy compounds (X—R₁—N═NO—R₂—Y) or hydrazo compounds (X—R₁ —NH—NH—R₂—Y). Particular examples of such azo compounds (or derivatives thereof) include Methyl Violet, Methyl Yellow, Methyl Orange, Methyl Red, and Methyl Green. For instance, Methyl Violet undergoes a transition from yellow to blue-violet at a pH range of about 0 to 1.6, Methyl Yellow undergoes a transition from red to yellow at a pH range of about 2.9 to 4.0, Methyl Orange undergoes a transition from red to yellow at a pH range of about 3.1 to 4.4, and Methyl Red undergoes a transition from red to yellow at a pH range of about 4.2 to 6.3.

Arylmethanes (e.g., diarylmethanes and triarylmethanes) constitute still another class of suitable pH-sensitive colorants. Triarylmethane leuco bases, for example, have the following general structure:

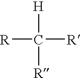

wherein R, R′, and R″ are independently selected from substituted and unsubstituted aryl groups, such as phenyl, naphthyl, anthracenyl, etc. The aryl groups may be substituted with functional groups, such as amino, hydroxyl, carbonyl, carboxyl, sulfonic, alkyl, and/or other known functional groups. Examples of such triarylmethane leuco bases include Leucomalachite Green, Pararosaniline Base, Crystal Violet Lactone, Crystal Violet Leuco, Crystal Violet, Cl Basic Violet 1, Cl Basic Violet 2, Cl Basic Blue, Cl Victoria Blue, N-benzoyl leuco-methylene, etc. Likewise suitable diarylmethane leuco bases may include 4,4′-bis(dimethylamino) benzhydrol (also known as “Michler's hydrol”), Michler's hydrol leucobenzotriazole, Michler's hydrol leucomorpholine, Michler's hydrol leucobenzenesulfonamide, etc. In one particular embodiment, the colorant is Leucomalachite Green Carbinol (Solvent Green 1) or an analog thereof, which is normally colorless and has the following structure:

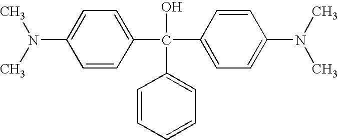

Under acidic conditions, one or more free amino groups of the Leucomalachite Green Carbinol form may be protonated to form Malachite Green (also known as Aniline Green, Basic Green 4, Diamond Green B, or Victoria Green B), which has the following structure:

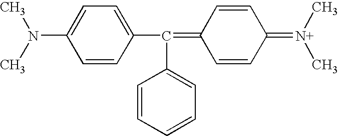

Malachite Green typically exhibits a transition from yellow to blue-green over a pH range 0.2 to 1.8. Above a pH of about 1.8, malachite green turns a deep green color.

Still other suitable pH-sensitive colorants that may be employed in the array include Congo Red, Litmus (azolitmin), Methylene Blue, Neutral Red, Acid Fuchsin, Indigo Carmine, Brilliant Green, Picric acid, Metanil Yellow, m-Cresol Purple, Quinaldine Red, Tropaeolin OO, 2,6-dinitrophenol, Phloxine B, 2,4-dinitrophenol, 4-dimethylaminoazobenzene, 2,5-dinitrophenol, 1-Naphthyl Red, Chlorophenol Red, Hematoxylin, 4-nitrophenol, nitrazine yellow, 3-nitrophenol, Alkali Blue, Epsilon Blue, Nile Blue A, universal indicators, and so forth. For instance, Congo Red undergoes a transition from blue to red at a pH range of about 3.0 to 5.2, Litmus undergoes a transition from red to blue at a pH range of about 4.5 to 8.3, and Neutral Red undergoes a transition from red to yellow at a pH range of about 11.4 to 13.0.

In addition to pH, other mechanisms may also be wholly or partially responsible for inducing a color change in the colorant. For example, many microorganisms (e.g., bacteria and fungi) produce low molecular weight iron-complexing compounds in growth media, which are known as “siderophores.” Metal complexing colorants may thus be employed in some embodiments, that undergo a color change in the presence of siderophores. One particularly suitable class of metal complexing colorants are aromatic azo compounds, such as Eriochrome Black T, Eriochrome Blue SE, Eriochrome Blue Black B, Eriochrome Cyanine R, Xylenol Orange, Chrome Azurol S, carminic acid, etc. Still other suitable metal complexing colorants may include Alizarin Complexone, Alizarin S, Arsenazo III, Aurintricarboxylic acid, 2,2′-Bipyidine, Bromopyrogallol Red, Calcon (Eriochrome Blue Black R), Calconcarboxylic acid, Chromotropic acid, disodium salt, Cuprizone, 5-(4-Dimethylamino-benzylidene)rhodanine, Dimethylglyoxime, 1,5-Diphenylcarbazide, Dithizone, Fluorescein Complexone, Hematoxylin, 8-Hydroxyquinoline, 2-Mercaptobenzothiazole, Methylthymol Blue, Murexide, 1-Nitroso-2-naphthol, 2-Nitroso-1-naphthol, Nitroso-R-salt, 1,10-Phenanthroline, Phenylfluorone, Phthalein Purple, 1-(2-Pyridylazo)-naphthol, 4-(2-Pyridylazo)resorcinol, Pyrogallol Red, Sulfonazo III, 5-Sulfosalicylic acid, 4-(2-Thiazolylazo)resorcinol, Thorin, Thymolthalexon, Tiron, Tolurnr-3,4-dithiol, Zincon, and so forth. It should be noted that one or more of the pH-sensitive colorants referenced above may also be classified as metal complexing colorants.

Of course, the colorants need not be capable of independently differentiating between particular microorganisms. In this regard, colorants may also be employed that exhibit a detectable color change in the presence of a broad spectrum of microorganisms. Solvatochromic colorants, for instance, are believed to exhibit a detectable color change in the presence of a broad spectrum of microorganisms. More specifically, solvatochromic colorants may undergo a color change in a certain molecular environment based on solvent polarity and/or hydrogen bonding propensity. For example, a solvatochromic colorant may be blue in a polar environment (e.g., water), but yellow or red in a non-polar environment (e.g., lipid-rich solution). The color produced by the solvatochromic colorant depends on the molecular polarity difference between the ground and excited state of the colorant.

Merocyanine colorants (e.g., mono-, di-, and tri-merocyanines) are one example of a type of solvatochromic colorant that may be employed in the present invention. Merocyanine colorants, such as merocyanine 540, fall within the donor—simple acceptor colorant classification of Griffiths as discussed in “Colour and Constitution of Organic Molecules” Academic Press, London (1976). More specifically, merocyanine colorants have a basic nucleus and acidic nucleus separated by a conjugated chain having an even number of methine carbons. Such colorants possess a carbonyl group that acts as an electron acceptor moiety. The electron acceptor is conjugated to an electron donating group, such as a hydroxyl or amino group. The merocyanine colorants may be cyclic or acyclic (e.g., vinylalogous amides of cyclic merocyanine colorants). For example, cyclic merocyanine colorants generally have the following structure:

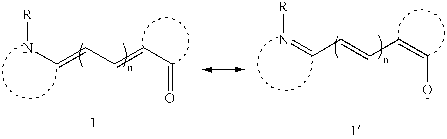

wherein, n is any integer, including 0. As indicated above by the general structures 1 and 1′, merocyanine colorants typically have a charge separated (i.e., “zwitterionic”) resonance form. Zwitterionic colorants are those that contain both positive and negative charges and are net neutral, but highly charged. Without intending to be limited by theory, it is believed that the zwitterionic form contributes significantly to the ground state of the colorant. The color produced by such colorants thus depends on the molecular polarity difference between the ground and excited state of the colorant. One particular example of a merocyanine colorant that has a ground state more polar than the excited state is set forth below as structure 2.

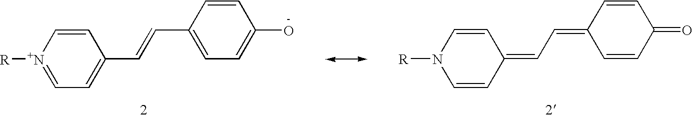

The charge-separated left hand canonical 2 is a major contributor to the ground state whereas the right hand canonical 2′ is a major contributor to the first excited state. Still other examples of suitable merocyanine colorants are set forth below in the following structures 3-13.

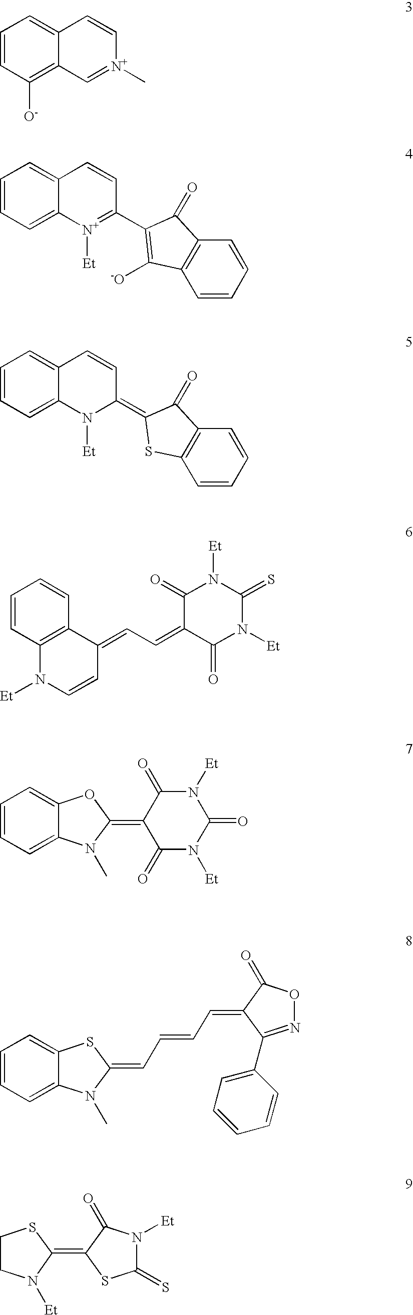

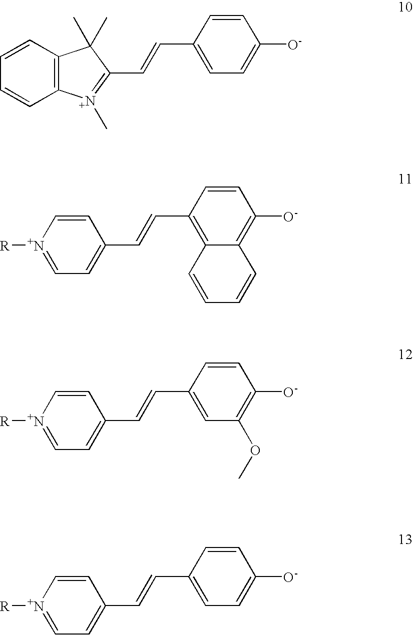

wherein, “R” is a group, such as methyl, alkyl, aryl, phenyl, etc.

Indigo is another example of a suitable solvatochromic colorant for use in the present invention. Indigo has a ground state that is significantly less polar than the excited state. For example, indigo generally has the following structure 14:

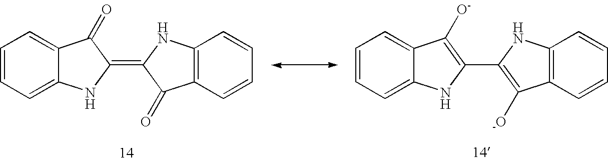

The left hand canonical form 14 is a major contributor to the ground state of the colorant, whereas the right hand canonical 14′ is a major contributor to the excited state.

Other suitable solvatochromatic colorants that may be used in the present invention include those that possess a permanent zwitterionic form. That is, these colorants have formal positive and negative charges contained within a contiguous π-electron system. Contrary to the merocyanine colorants referenced above, a neutral resonance structure cannot be drawn for such permanent zwitterionic colorants.

Exemplary colorants of this class include N-phenolate betaine colorants, such as those having the following general structure:

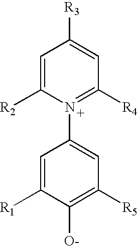

wherein R₁-R₅ are independently selected from the group consisting of hydrogen, a nitro group (e.g., nitrogen), a halogen, or a linear, branched, or cyclic C₁ to C₂₀ group (e.g., alkyl, phenyl, aryl, pyridinyl, etc.), which may be saturated or unsaturated and unsubstituted or optionally substituted at the same or at different carbon atoms with one, two or more halogen, nitro, cyano, hydroxy, alkoxy, amino, phenyl, aryl, pyridinyl, or alkylamino groups. For example, the N-phenolate betaine colorant may be 4-(2,4,6-triphenylpyridinium-1-yl)-2,6-diphenylphenolate (Reichardt's dye) having the following general structure 15:

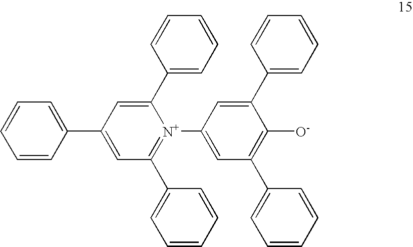

Reichardt's dye shows strong negative solvatochromism and may thus undergo a significant color change from blue to colorless in the presence of bacteria. That is, Reichardt's dye displays a shift in absorbance to a shorter wavelength and thus has visible color changes as solvent eluent strength (polarity) increases. Still other examples of suitable negatively solvatochromic pyridinium N-phenolate betaine colorants are set forth below in structures 16-23:

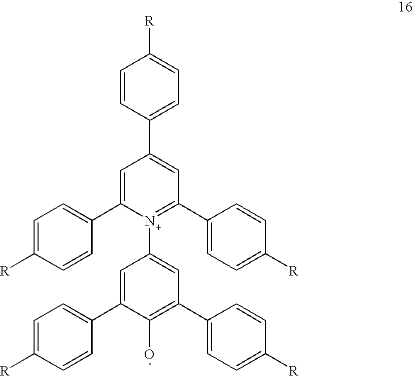

wherein, R is hydrogen, —C(CH₃)₃, —CF₃, or C₆F₁₃.

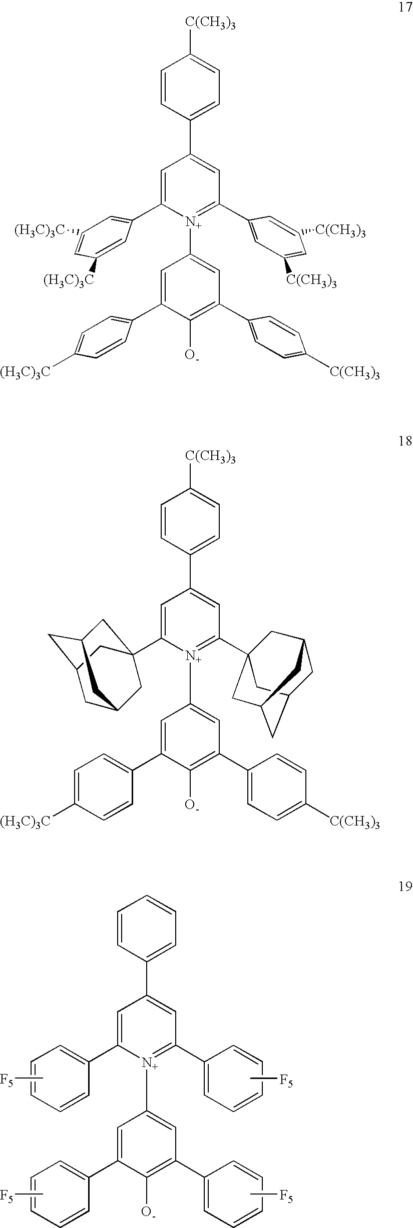

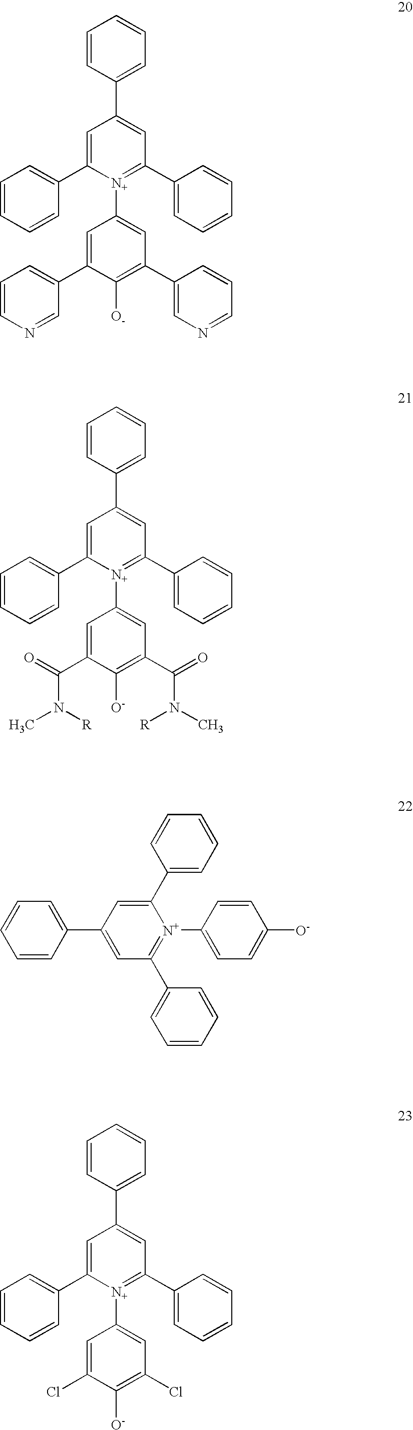

Still additional examples of colorants having a permanent zwitterionic form include colorants having the following general structure 24:

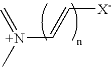

wherein, n is 0 or greater, and X is oxygen, carbon, nitrogen, sulfur, etc. Particular examples of the permanent zwitterionic colorant shown in structure 24 include the following structures 25-33.

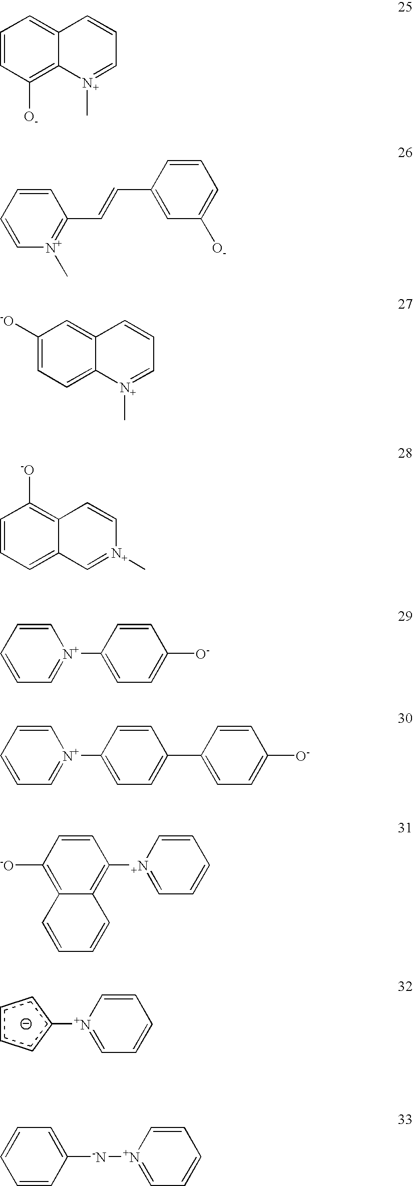

Still other suitable solvatochromic colorants may include, but are not limited to 4-dicyanmethylene-2-methyl-6-(p-dimethylaminostyryl)-4H-pyran (DCM); 6-propionyl-2-(dimethylamino)naphthalene (PRODAN); 9-(diethylamino)-5H-benzo[a]phenox-azin-5-one (Nile Red); 4-(dicyanovinyl)julolidine (DCVJ); phenol blue; stilbazolium colorants; coumarin colorants; ketocyanine colorants; N,N-dimethyl-4-nitroaniline (NDMNA) and N-methyl-2-nitroaniline (NM2NA); Nile blue; 1-anilinonaphthalene-8-sulfonic acid (1,8-ANS), and dapoxylbutylsulfonamide (DBS) and other dapoxyl analogs. Besides the above-mentioned colorants, still other suitable colorants that may be used include, but are not limited to, 4-[2-N-substituted-1,4-hydropyridin-4-ylidine)ethylidene]cyclohexa-2,5-dien-1-one, red pyrazolone colorants, azomethine colorants, indoaniline colorants, and mixtures thereof.

Although the above-referenced colorants are classified based on their mechanism of color change (e.g., pH sensitive, metal complexing, or solvatochromatic), it should be understood that the present invention is not limited to any particular mechanism for the color change. Even when a pH-sensitive colorant is employed, for instance, other mechanisms may actually be wholly or partially responsible for the color change of the colorant. For example, redox reactions between the colorant and microorganism may contribute to the color change.

TABLE 1
Exemplary Colorants and Their Corresponding Structure

| Colorant | Structure | |
|---|---|---|
| 4-[(1-methyl-4(1H)-pyri-dinylidene)ethyl-idene]-2,5-cyclo-hexadien-1-one hydrate | 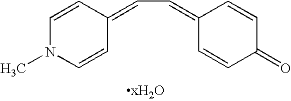 | |
| 3-Ethyl-2-(2-hydroxy-1-pro-penyl)benzothiazoliumchloride | 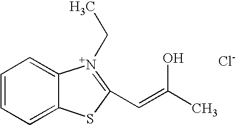 | |
| 1-Docosyl-4-(4-hy-droxystyryl)pyridiniumbromide | 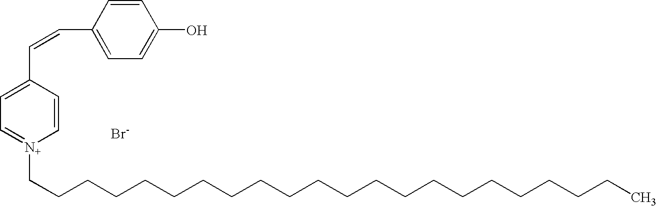 | |
| N,N-Dimethylindoaniline | 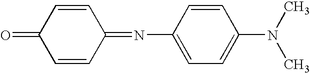 | |
| Quinalizarin | 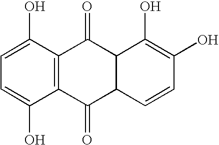 | |
| Merocyanine 540 | 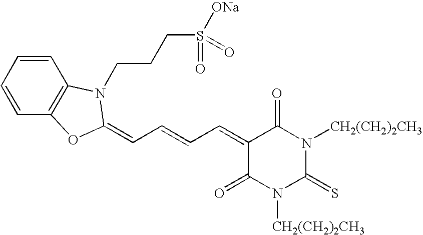 | |
| Eriochrome Blue SE | 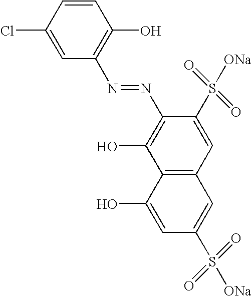 | |
| Phenol Red | 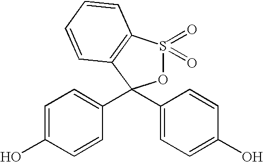 | |
| Nile Blue A | 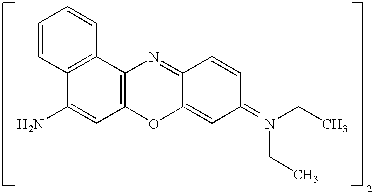 | 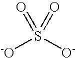 |

| 1-(4-Hydroxy-phenyl)-2,4,6-tri-phenylpyridinium hydroxideinnersalt hydrate | 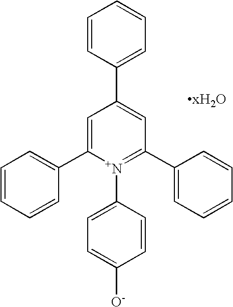 | |
|---|---|---|
| Azomethine-H monosodiumsalt hydrate | 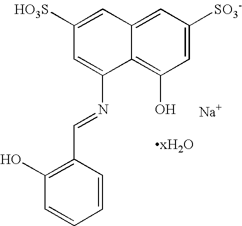 | |
| Indigo carmine | 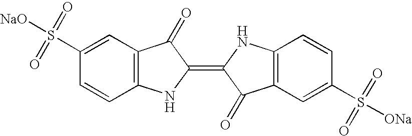 | |
| Methylene Violet | 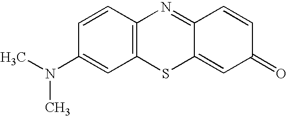 | |
| Eriochrome Blue Black B | 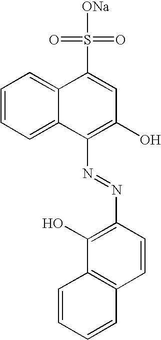 | |
| Methylene Blue | 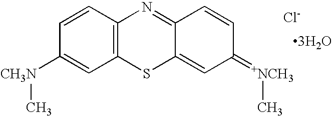 | |
| Nile Red | 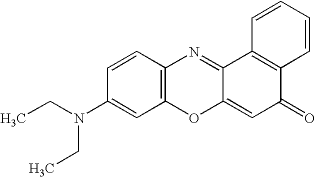 | |
| Trypan Blue | 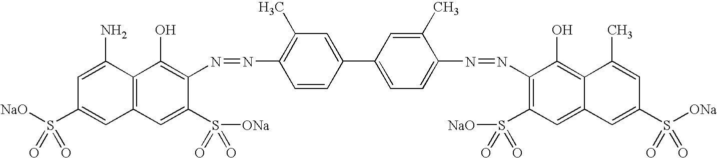 | |
| Safranin O | 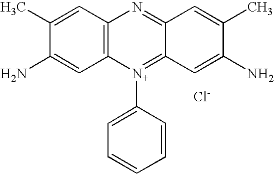 | |
| Crystal Violet | 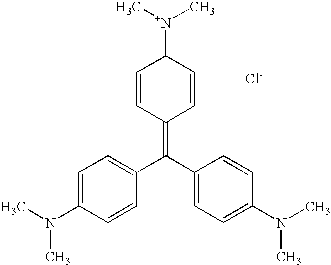 | |
| Methyl Orange | 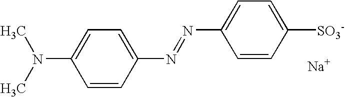 | |
| Chrome Azurol S | 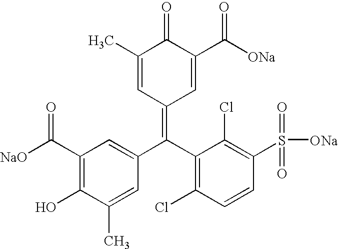 | |
| Leucocrystal violet | 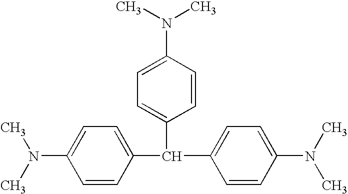 | |
| Leucomalachite Green | 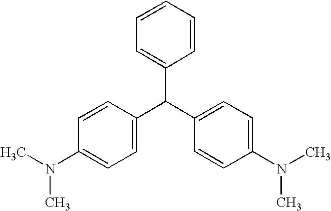 | |
| Leuco xylene cyanole FF | 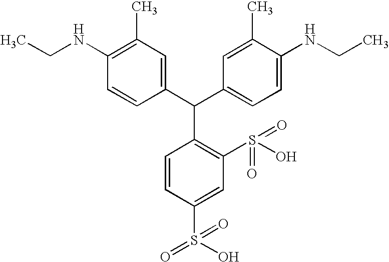 | |
| 4,5-Dihydroxy-1,3-benzene-disulfonicacid disodiumsalt monohydrate | 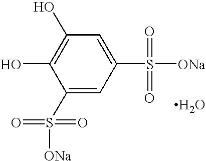 | |
| 5-Cyano-2-[3-(5-cyano-1,3-di-ethyl-1,3-di-hydro-2H-benzimidazol-2-yl-idene)-1-propenyl]-1-eth-yl-3-(4-sulfo-butyl)-1H-benzimidazoliumhydroxide inner salt | 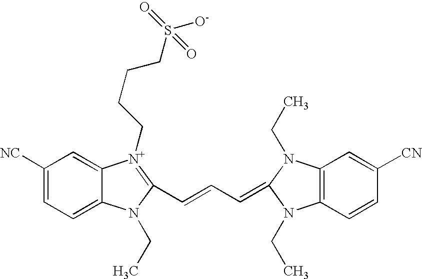 | |
| Acid Green 25 | 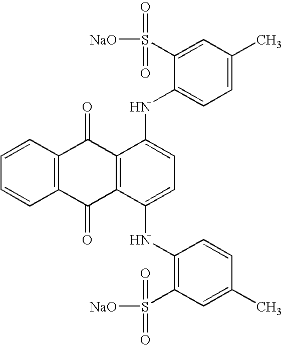 | |
| Bathophenanthrolinedisulfonicaciddisodium salt trihydrate | 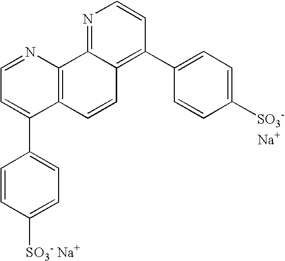 | |
| Carminic Acid | 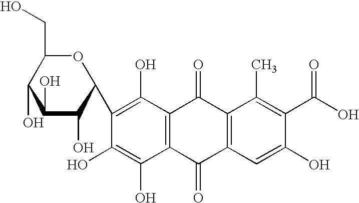 | |
| Celestine Blue | 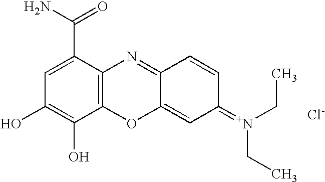 | |
| Hematoxylin | 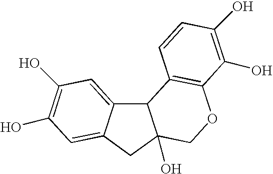 | |
| Bromophenol Blue | 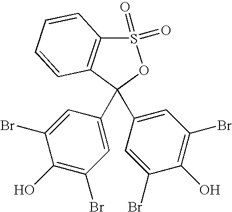 | |
| Bromothymol blue | 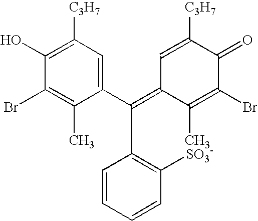 | |
| Rose Bengal | 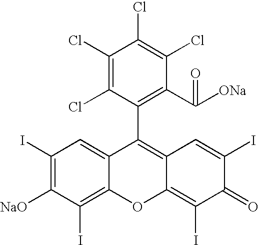 | |
| Universal indicator 0–5 | Not available | |
| Universal indicator 3–10 | Not available | |
| Alizarin Complexone | 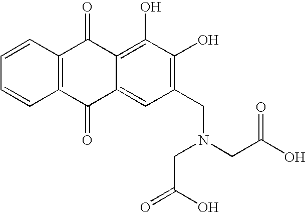 | |
| Alizarin Red S | 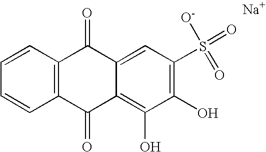 | |
| Purpurin | 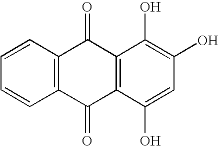 | |
| Alizarin | 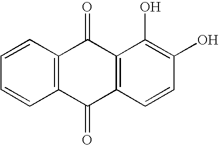 | |
| Emodin | 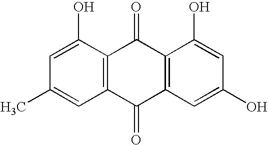 | |
| Amino-4-hydroxy-anthraquinone | 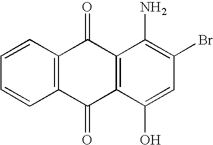 | |
| Nuclear Fast Red | 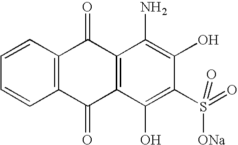 | |
| Chlorophenol Red | 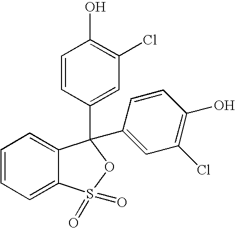 | |
| Remazol Brilliant Blue R | 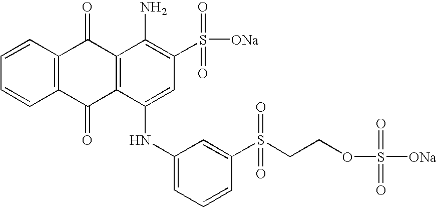 | |
| Procion Blue HB | 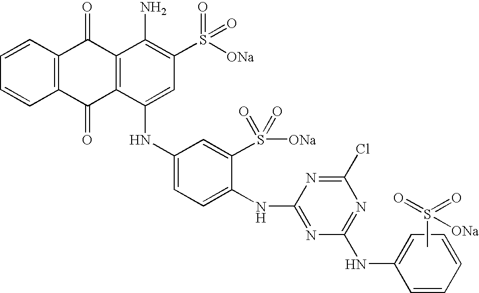 | |
| Phenolphthalein | 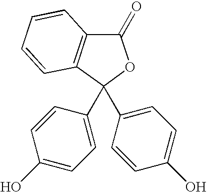 | |
| Ninhydrin | 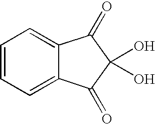 | |
| Nitro blue tetrazolium | 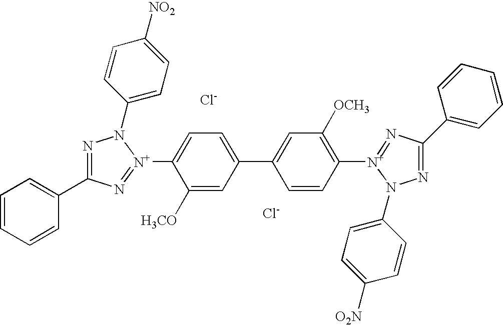 | |
| Orcein | 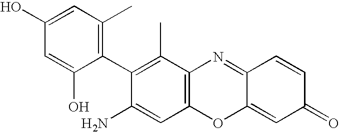 | |
| Celestine blue | 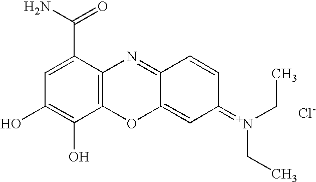 | |
| Tetra Methyl-para-phenylenediamine (TMPD) | 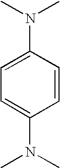 | |
| 5,10,15,20-Tetra-kis(pentafluoro-phenyl)porphyriniron(III) chloride | 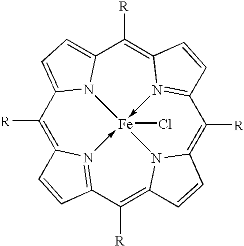 | 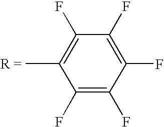 |

The color change inherent in the skin coating with indicator may be considered as a visual indicator with the user visually observing a color change as a signal that infection or microbial contamination is present, or the color change could also be measured electronically. Such measurements could be conducted using an optical device or other spectroscopic methods known to those skilled in the art to measure changes in color such as spectrophotometers and spectrodenitometers. The instruments measure color space (as described in “Pocket guide to digital printing” (1997) by Frank Cost, Delmar Publishers Inc., at page 144), the most widely used color space is CIELAB. This defines three variables, L*, a*, and b*, that have the following meaning:

L*=lightness, ranging from 0=dark and 100=light.

A*=red/green axis, ranging approximately from −100 to 100. Positive values are reddish and negative values are greenish.

B*=yellow/blue axis, ranging from approximately from −100 to 100. Positive values are yellowish and negative values are blueish.

Because CIELAB color space is somewhat uniform, a single number can be calculated that represents the difference between two colors as perceived by the human being. This difference is termed ΔE and is calculated by taking the square root of the sum of the squares of the three differences (ΔL*, Δa*, and Δb*) between the two colors (i.e. starting color and after color change).

In CIELAB color space, each ΔE unit is roughly a just-noticeable difference between the two colors. A difference of ΔE is clearly visible to a human eye. It is preferred that the microbial indicator herein gives a measurable change in color of ΔE>3.

The composition containing the dye indicator may be packaged in a “kit” form for use in medical facilities and bundled with the appropriate skin prep solution for ease of use and the convenience of the medical personnel.

The following examples show the efficacy of the invention.

### EXAMPLE 1

### Reichardt's Dye

Reichardt's dye (from Sigma-Aldrich Chemical Co. Inc., Milwaukee Wis.) was mixed into 2 grams of InteguSeal® skin sealant (Medlogic Global Ltd., Cornwall, UK) containing an extra 0.2 gram of tributyl o-acetylcitrate placticizer (from Sigma-Aldrich) to give a deep blue solution with 200 ppm concentration of the dye. A drop (25 mg) of the mixture was then placed onto a microscope glass slide (5 cm×7.5 cm) and spread out using a glass rod to give thin coating smear (3 cm×2 cm). After complete cure had occurred (5 minutes) the cured film was exposed to a suspension of S. aureus (gram positive bacteria) at 10⁶ CFU/mL (colony forming units). 100 μL of this suspension was placed onto a spot on the cured film and the area observed. In less than 10 seconds the area in contact with the bacteria suspension decolorized, visually indicating the contamination. When a 100 μL sample of the control media broth or water alone was placed on the sealant no color discharge was observed.

### EXAMPLE 2

### Chrome Azurol S

A 2 gram blue-purple solution of 300 ppm Chrome Azurol S (from Sigma-Aldrich) in InteguSeal® skin sealant was prepared to give a 300 ppm concentration of the dye. A drop 25 mg of the mixture was placed onto a glass slide and spread using a glass rod to give a thin smear. The sealant was allowed to fully cure (5 minutes). After this time 100 μL suspension of S. aureus at 10⁶ CFU/mL was placed on the cure sealant and then visually observed for a color change. A red color developed within 5 seconds where the bacteria was in contact with the film.

### EXAMPLE 3

### Phenol Red

A 2 gram solution of 300 ppm Phenol Red (from Sigma-Aldrich) in InteguSeal® skin sealant was prepared by mixing the ingredients to give a pale pink-gray liquid. 25 mg of the mixture was then placed onto a glass slide and spread out using a glass rod to give a thin coating smear on the glass. After the mixture fully cured (5 minutes) 100 μL of suspension of S. aureus bacteria at 10⁶ CFU/mL was placed onto the cured sealant and visually observed for any color change. A bright red color developed, in less than 5 seconds, where the liquid was in contact with the film. No color change or development was observed when the color media or water was placed on the sealant film.

### EXAMPLE 4

### Eriochrome Blue Black B

A 2 gram sample of 300 ppm Eriochrome Blue Black B (from Sigma-Aldrich) in InteguSeal® skin sealant was prepared by mixing the ingredients to give a gray-blue mixture. 25 mg of the mixture was placed on a glass slide and spread with a glass rod to give a thin smear. The film was allowed to fully cure (5 minutes) and then 100 μL of S. aureus suspension at 10⁶ CFU/mL was applied to the cured film and observed for a color change. In less than 5 seconds the film color was discharged to leave a colorless spot where the liquid was in direct contact with the film. No color change was observed when control media or water was applied to the film.

### EXAMPLE 5

### Phenol Red with E. coli

A 2 gram solution of 300 ppm Phenol Red (from Sigma-Aldrich) in InteguSeal® skin sealant was prepared by mixing the ingredients to give a pale pink-gray liquid. 25 mg of the mixture was then placed onto a glass slide and spread out using a glass rod to give a thin coating smear on the glass. After the mixture fully cured (5 minutes) 100 μL of suspension of E. coli bacteria at 10⁵ CFU/mL was placed onto the cured sealant and visually observed for any color change. A bright red color developed in less than 5 seconds where the liquid was in contact with the film. No color change or development was observed when the color media or water was placed on the sealant film.

### EXAMPLE 6

### Reichardt's Dye with E. coli and Also A. Niger

Reichardt's dye (from Sigma-Aldrich) was mixed into 2 grams of InteguSeal® skin sealant containing an extra 0.2 gram of tributyl o-acetylcitrate placticizer (from Sigma-Aldrich) to give a deep blue solution with 200 ppm concentration of the dye. A drop (25 mg) of the mixture was then placed onto a microscope glass slide (5 cm×7.5 cm) and spread out using a glass rod to give thin coating smear (3 cm×2 cm). After complete cure had occurred (5 minutes) the cured film was exposed to a suspension of E. coli (gram negative bacteria) at 10⁵ CFU/mL (colony forming units). 100 μL of this suspension was placed onto a spot on the cured film and the area observed. In less than 10 seconds the area in contact with the bacteria suspension decolorized, visually indicating the contamination. When a 100 μL sample of the control media broth or water alone was placed on the sealant no color discharge was observed. On a separate untouched part of the cured film was placed 100 μL suspension of A. niger (mold) at 10⁵ CFU/mL and the area again observed. In less than 10 seconds the Reichardt's dye had decolorized where the mold suspension was in contact with the film.

### EXAMPLE 7

### Microbial Indicator in Other Curable Resins

Phenol red was dissolved in 3 other curable resins at a concentration of 300 ppm and tested with E. coli as described in example 5 above. The resins tried were:
Elmer's glue-all (Elmer's Products Inc., Columbus Ohio)
Contact cement (DAP Weldwood Inc., Dayton, Ohio)
Gelatin USP (unflavored. The Kroger Co., Cincinnati, Ohio)
   100 μL suspension of E. coli was then placed on the cured film and visually observed. In all three resins the area in direct contact with the bacteria suspension turned red.

### EXAMPLE 8

### Color Change Measurements

Although in each of the examples described above the color change when in contact with microbes was clearly visible it would also be possible to measure this color change with an optical color change meter or sensor. This was conduced on Examples 1, 2, and 3 by illustration. The color change was measured using a spectrometer (Minolta cm-2600d. Minolta Co., Japan) and the reading obtained by measuring the film area before and after exposure to microbes. The reading was recorded in units of ΔE.

### Example 1=ΔE of 32.

### Example 2=ΔE of 27.

### Example 3=ΔE of 35.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A curable coating comprising at least one microbial indicator.

2. The coating of claim 1 where there is at least one microbial indicator and other non-indicating color dye or colorant.

3. The coating of claim 1 where the microbial indicator gives a visible color change.

4. The coating of claim 1 where the microbial indicator gives a measurable change in color of ΔE>3.

5. The coating of claim 1 wherein said indicator is present in an amount between about 1 and 1000 ppm.

6. The coating of claim 1 wherein said indicator is present in an amount between about 50 and 800 ppm.

7. The coating of claim 1 wherein said indicator is present in an amount between about 100 and 500 ppm.

8. The coating of claim 1 which comprises a vinylic monomer, latex, polyvinylalcohol, or gelatin.

9. The coating of claim 8 where the coating comprises a vinylic monomer, where the vinylic monomer is cyanoacrylate.

10. The coating of claim 9 where the cyanoacrylate comprises a 2-alkyl cyanoacrylate where the alkyl group is a C₁ to C₈ hydrocarbon which is straight chain, branched chain, or cyclic.

11. The coating of claim 1 where the microbial indicator can indicate the presence of bacteria, molds, yeasts or viruses.

12. The coating of claim 1 wherein said indicator is a pH sensitive colorant, a phthalein, anthraquinone, arylmethane, aromatic azo, a metal complexing colorant or a solvatochromic colorant.

13. The coating of claim 12 where the indicator is a pH sensitive colorant microbial indicator which is phenol red.

14. The coating of claim 12 where the indicator is a phthalein microbial indicator which is phenolphthalein.

15. The coating of claim 12 where the indicator is an anthraquinone microbial indicator which is remazol brilliant blue R.

16. The coating of claim 12 where the indicator is an arylmethane microbial indicator which is chrome azurol S.

17. The coating of claim 12 where the indicator is an aromatic azo microbial indicator which is eriochrome blue black B.

18. The coating of claim 12 where the indicator is a metal complexing colorant microbial indicator is alizarin complexone.

19. The coating of claim 1 where the indicator is a the solvatochromic colorant microbial indicator which is Reichardt's dye.

20. The coating of claim 1 wherein said indicator gives a visual change in color in less than 20 minutes.
